(19) United States
(12) Patent Application Publication
(10) Pub. No.: US 2015/0057452 A1
(43) Pub. Date: Feb. 26, 2015

(54) SELECTIVE ANDROGEN RECEPTOR MODULATORS

(71) Applicant: Catylix, Inc., Burbank, CA (US)

(72) Inventors: James David Rozzell, Burbank, CA (US); Raymond McCague, Cambridge (GB)

(73) Assignee: Catylix, Inc., Burbank, CA (US)

(21) Appl. No.: 14/390,735

(22) PCT Filed: Apr. 4, 2013
(86) PCT No.: PCT/US2013/035242

§ 371 Date: Oct. 3, 2014

Related U.S. Application Data
(60) Provisional application No. 61/620,042, filed on Apr. 4, 2012.

(51) Int. Cl. C07C 255/60; C07C 317/46; C07C 315/02; C07D 233/86; C07C 17/32; C07C 253/14; C07C 209/74; C07C 201/08; C07C 209/36; C07C 253/30; C07C 319/20

(52) U.S. Cl. CPC C07C 255/60; C07C 253/30; C07C 317/46; C07C 315/02; C07D 233/86; C07C 319/20; C07C 253/14; C07C 209/74; C07C 201/08; C07C 209/365; C07C 17/32
USPC 548/319.5; 558/414; 558/413

(57) ABSTRACT

Compounds having therapeutic potential as androgen receptor modulators, and methods of making such compounds, are provided. The compounds are structurally related to bicalutamide but bear at least one difluoromethyl or C₂ to C₅ perfluoroalkyl group instead of a trifluoromethyl group.

### BACKGROUND OF THE INVENTION

Selective androgen receptor modulators are compounds that bind to androgen receptors in some selective way to modulate the level of activation of the androgen receptor whilst displacing the binding of any endogenous androgen such as testosterone. Selective androgen receptor modulators, to the extent that they act as antagonists of the androgen receptor, have therapeutic potential for treating diseases that are aggravated by androgens, such as benign prostatic hypertrophy, prostate cancer, and over masculinity in women. To the extent that they act as agonists for the androgen receptor, they have therapeutic potential for treating ailments associated with a deficiency of androgens in men, such as hypogonadism, and frailty and muscle wasting as androgen levels decline with advancing age.

Typical archetypal androgen receptor modulators are flutamide and bicalutamide; their structures are shown below:

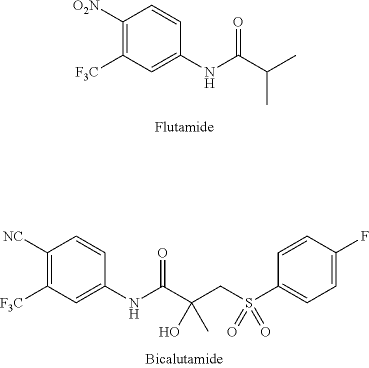 [SMILES: CC(C)C(=O)Nc1ccc([N+](=O)[O-])c(C(F)(F)F)c1 ; CC(O)(CS(=O)(=O)c1ccc(F)cc1)C(=O)Nc1ccc(C#N)c(C(F)(F)F)c1]

These compounds are anti-androgens and are primarily useful for treatment of prostate cancer. Flutamide is one of the compounds described in U.S. Pat. No. 3,995,060 and is marketed by Schering-Plough under such trade names as Eulexin and Flutamin. It has largely been superseded by bicalutamide, which has a better side-effect profile. Bicalutamide was launched by AstraZeneca in 1995 under the brand names Casodex and Cosudex. These compounds and various analogues in development comprise an acylated aniline bearing either a nitro or a nitrile substituent, and a trifluoromethyl substituent.

The trifluoromethyl substituent is sometimes found in therapeutic compounds, typically as a substituent on a phenyl ring, but synthetic methodologies that access such structures are limited. Compounds bearing other trifluoroalkyl groups are rare and more difficult to make. One route to trifluoromethylphenyl compounds is the use of chemical intermediates that already contain the trifluoromethyl group, such as α,α,α-trifluorotoluene (also known as trifluoromethylbenzene and benzotrifluoride). This intermediate can be produced from toluene by chlorination of toluene to α,α,α-trichlorotoluene (benzotrichloride) and then substitution of fluorine for chlorine by a displacement reaction with hydrogen fluoride, as reviewed in D. P Curran et al, Top. Curr. Chem., 1999, 206, 79-105. Once supplied with benzotrifluoride or a related trifluoromethylphenyl intermediate, additional substitution can be made onto the phenyl ring by standard organic synthetic chemistry practises known to a medicinal chemist skilled in the art.

There is even less synthetic accessibility to corresponding intermediates having different fluoroalkyl groups, such as pentafluoroethyl and difluoromethyl. To access such compounds would require use of different chemistry, and since such chemistry is not generally practised by medicinal chemists skilled in the art, and since the requisite starting materials bearing such different fluoroalkyl groups are often commercially unavailable, the utility of fluoroalkyl groups other than trifluoromethyl in medicinal chemistry is generally overlooked.

There have been few initiatives in the prior art of medicinal chemistry concerning the use of fluoroalkyl groups other than trifluoromethyl. By introducing a fluoroalkyl substituent from a condensation reaction using a fluoroalkylcarboxylic acid, various fluoroalkyl substituents can be introduced if the appropriate carboxylic acid is available. For example, Merrell Dow's EP529568 describes pentafluoroethylpeptides derived from pentafluoropropionic acid as elastase inhibitors. Merck's U.S. Pat. No. 3,962,262 reports 1,8-naphthyridine compounds as bronchodilating agents, again derived from condensation reactions on pentafluoropropionic acid. In U.S. Pat. No. 5,092,247, a 3-difluoromethylpyrazole fungicide agent incorporates the difluoromethyl group from ethyl difluoroacetate. However, synthesis starting from a fluoroalkylcarboxylic acid does not usually apply to making fluoroalkylbenzene derivatives. By a different method, U.S. Pat. No. 4,604,406 describes perfluoroalkyl naphthalenes as agents for treating diabetic complications, made by coupling the iodonaphthalene with the iodoperfluoroalkane and copper. Likewise, Merck's U.S. Pat. No. 5,602,152 describes a set of benzoxapine as potassium channel activators, and includes an example with a pentafluoroethyl group borne on a phenyl ring. Amongst several analogues, pentafluoroethyl-phenyl compounds appear as anti-angiogenesis agents in US2006194848. Also, G. D. Searle's U.S. Pat. No. 6,458,803 reports CETP inhibitors for treating atherosclerosis and includes examples with a pentafluoroethyl or heptafluoropropyl substituent attached to a phenyl ring.

However, these prior disclosures of the use of fluoroalkyl compounds other than trifluoromethyl in medicinal chemistry have not been enabled or exemplified in the field of selective androgen receptor modulators; and the specifically enabling disclosures only refer to compounds bearing only a trifluoromethyl group.

In the case of selective androgen receptor modulator MDV3100, which is described in US2007254933, all the compounds contain the same cyanotrifluoromethylphenyl moiety, so use of other fluoroalkyl groups was not considered. In the case of the selective androgen receptor modulator Ostarine (GTx-024) as described in U.S. Pat. No. 6,569,896, the relevant phenyl group substituent is claimed to be either -iodo, -trifluoromethyl, -bromo, -chloro, or trialkylstannyl-, so fluoroalkyl groups other than trifluoromethyl have not been considered. A subsequent patent application, WO2008127717, directed to the use of selective androgen receptor modulators for treating diabetes, identifies trifluoromethyl as the only trifluoroalkyl substituent considered.

Of the archetypal selective androgen receptor modifiers, bicalutamide is described in U.S. Pat. No. 4,636,505. Although the claims in the '505 patent recite fluoro substitutions in addition to trifluoromethyl, i.e., perfluoroalkyl up to four carbon atoms, the reference does not provide an enabling disclosure for fluoro-substitutions other than trifluoromethyl. The '505 patent does not describe any appreciation of any advantages that might be possessed by other fluoroalkyl analogues.

Flutamide is described in U.S. Pat. No. 3,995,060. Although “polyfluoroloweralkyl”-substituted compounds are claimed (the term is defined to include difluoromethyl, trifluoromethyl, α,α-difluoroethyl, and β,β,β-trifluoroethyl), the only examples listed as completed compounds are all trifluoromethyl analogues, with the exception of an example of an intermediate having an α,α-difluoroethyl group.

With regards to the synthesis of existing androgen receptor modulators such as bicalutamide, the key commercial raw intermediate is 4-amino-2-trifluoromethylbenzonitrile. This compound may be manufactured as described in the European Patent Application EP00028921979 by way of cyanide displacement of 2-bromo-5-aminobenzotrifluoride, or, as described in Patent US30120581961, using a synthetic sequence via 2-amino-5-chlorobenzotrifluoride by diazotisationcyanide to introduce the nitrile group, and then displacement of the chloro- with ammonia. Either way, the syntheses may be traced back to benzotrifluoride and, given that benzotrifluoride is made from toluene, it is not obvious to those skilled in the art how selective androgen receptor modulators with higher fluoroalkyl groups such as pentafluoroethyl might be synthesised in any method suitable for manufacturing a selective androgen receptor modulator compound.

### SUMMARY OF THE INVENTION

The present invention provides novel compounds bearing certain fluoroalkyl groups, such as pentafluoroethyl and pentafluoropropyl that have utility for pharmaceutical research and development and potential therapeutic use as selective androgen receptor modulators, and processes for making them using fluoroalkylsilane reagents and suitable catalysts. In one aspect of the invention, a compound having the formula (1) is provided:

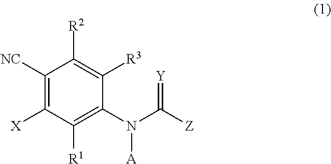

wherein:

X is difluoromethyl or C₂ to C₅ perfluoroalkyl

Y is oxygen or sulfur;

Z is selected from the group consisting of
(i) C₁ to C₆ alkyl, which may be linear or branched,
(ii) C₃ to C₆ cycloalkyl,
(iii) a five- or six-membered aryl group,
(iv) C₁ to C₆ alkoxy,
(v) C₃ to C₆ cycloalkoxy,
(vi) aryloxy wherein the aryl group is five- or six-membered,
(vii) amino,
(viii) alkylamino wherein the alkyl group is of 1 to 6 carbon atoms and may be cycloalkyl,
(ix) dialkylamino wherein both alkyl groups are of 1 to 6 carbon atoms, and either or both may be cycloalkyl,
(x) arylamino wherein the aryl group is five- or six-membered,
(xi) diarylamino wherein both aryl groups are five- or six-membered,
(xii) arylalkylamino wherein the aryl group is five- or six-membered and the alkyl group is of 1 to 6 carbon atoms and may be cycloalkyl, and

wherein in each case (i) to (xii) said alkyl, cycloalkyl, alkoxy or aryl group optionally bears one or more groups selected from hydroxy, alkoxy (—OR), aryloxy (—OAr), arylthio (—SAr), arylsulfoxide (—SOAr), and arylsulfone (—SO₂Ar), wherein each R group is independently selected from C1 to C4 alkyl, each Ar group is independently a five- or six-membered ring, and each Rand each Ar group optionally bears one or more substituents selected from halogen, carboxamide (where the nitrogen atom optionally bears one or more C₁-C₄ alkyl groups), acylamino (where the acyl group contains 1 to 4 carbon atoms), and nitrile;

R¹, R², and R³ are, independently, selected from the group consisting of hydrogen, halogen, and C₁ to C₆ alkyl; and

A is either hydrogen, C₁ to C₄ alkyl, or acyl, or A together with Z forms a five- or six-member heterocyclic group.

For example, compounds analogous to bicalutamide, but having a difluoromethyl group (CHF₂) or C₂ to C₅ perfluoroalkyl group instead of a trifluoromethyl group, have the formula (1), wherein X is CHF₂ or C₂ to C₅ perfluoroalkyl; Y is oxygen, Z is 1-hydroxy-1-methyl-2-(p-fluorophenylsulfonyl)ethyl, and each of R¹, R², R³, and A is hydrogen.

### DETAILED DESCRIPTION OF THE INVENTION

This invention provides compounds useful for pharmaceutical investigation and potential therapeutic use as selective androgen receptor modulators. The compounds bear at least one difluoromethyl group or C₂ to C₅ perfluoroalkyl group. Improved therapeutic properties may result from any of (i) higher activity towards the androgen receptor, (ii) better selectivity for androgen receptors compared to binding to other proteins, or a better selectivity for androgen receptors in certain tissues, (iii) improved metabolism or disposition profile, and (iv) improved physical properties that may assist with the formulation of a therapeutic product.

Compounds of the present invention have the formula (1)

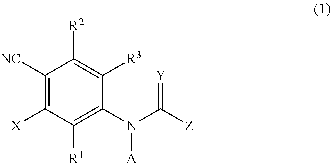

wherein:

X is difluoromethyl or C₂ to C₅ perfluoroalkyl;

Y is oxygen or sulfur;

Z is selected from the group consisting of
(i) C₁ to C₆ alkyl, which may be linear or branched,
(ii) C₃ to C₆ cycloalkyl,
(iii) a five- or six-membered aryl group,
(iv) C₁ to C₆ alkoxy,
(v) C₃ to C₆ cycloalkoxy,
(vi) aryloxy wherein the aryl group is five- or six-membered,
(vii) amino,
(viii) alkylamino wherein the alkyl group is of 1 to 6 carbon atoms and may be cycloalkyl,
(ix) dialkylamino wherein both alkyl groups are of 1 to 6 carbon atoms, and either or both may be cycloalkyl,
(x) arylamino wherein the aryl group is five- or six-membered,
(xi) diarylamino wherein both aryl groups are five- or six-membered,
(xii) arylalkylamino wherein the aryl group is five- or six-membered and the alkyl group is of 1 to 6 carbon atoms and may be cycloalkyl, and

wherein in each case (i) to (xii) said alkyl, cycloalkyl, alkoxy or aryl group optionally bears one or more groups selected from hydroxy, alkoxy (—OR), aryloxy (—OM, arylthio (—SAr), arylsulfoxide (—SOAr), and arylsulfone (—SO₂Ar), wherein each R group is independently selected from C1 to C4 alkyl, each Ar group is independently a five- or six-membered ring, and each Rand each Ar group optionally bears one or more substituents selected from halogen, carboxamide (where the nitrogen atom optionally bears one or more C₁-C₄ alkyl groups), acylamino (where the acyl group contains 1 to 4 carbon atoms), and nitrile;

R¹, R², and R³ are, independently, selected from the group consisting of hydrogen, halogen, and C₁ to C₆ alkyl; and

A is either hydrogen, C₁ to C₄ alkyl, or acyl, or A together with Z forms a five- or six-member heterocyclic group that optionally bears one or more groups selected from hydroxy, alkoxy (—OR), aryloxy (—OAr), arylthio (—SAr), arylsulfoxide (—SOAr), and arylsulfone (—SO₂Ar), wherein each R group is independently selected from C1 to C4 alkyl, each Ar group is independently a five- or six-membered ring, and each R and each Ar group optionally bears one or more substituents selected from halogen, carboxamide (where the nitrogen atom optionally bears one or more C₁-C₄ alkyl groups), acylamino (where the acyl group contains 1 to 4 carbon atoms), and nitrile. Also included are physiologically acceptable salts of compounds of formula (1).

In one embodiment, the five- or six-member heterocyclic group is selected from the group consisting of imidazolidine-2,4-dione, 2-thioxoimidazolidin-4-one, 5-thioxoimidazolidin-2-one, imidazolidin-2-one, imidazolidine-2-thione, pyrrolidin-2-one, pyrrolidine-2-thione, oxazolidin-2-one, oxazolidine-2-thione, oxazolidine-2,4-dione, 1,2,4-oxadiazolidine-3,5-dione, 1,2,4-triazolidine-3,5-dione, 3-thioxo-1,2,4-oxadiazolidin-5-one, 5-thioxo-1,2,4-triazolidin-3-one, tetrahydropyrimidin-2(1H)-one, tetrahydropyrimidin-2(1H)-thione, piperidin-2-one, 1,3-oxazinan-2-one, 1,3-oxazinane-2,4-dione, piperidine-2,6-dione, dihydropyrimidine-2,4(1H,3H)-dione, or 2-thioxotetrahydropyrimidine-4(1H)-one. Each such group may optionally bear one or more groups selected from hydroxy, alkoxy (—OR), aryloxy (—OM, arylthio (—SAr), arylsulfoxide (—SOAr), and arylsulfone (—SO₂Ar), wherein each R and Ar is as defined above.

Nonlimiting examples of such compounds include the following:

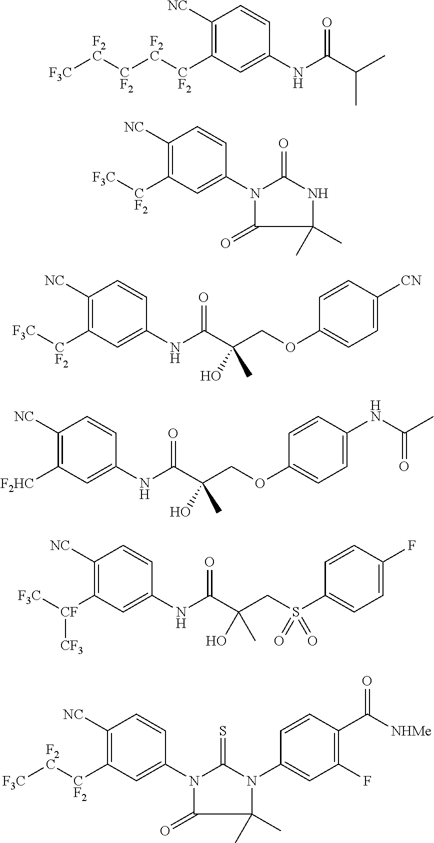

Such compounds are readily prepared using recently developed technology for introducing a fluoroalkyl group into an arene using copper-mediated fluoroalkylation, as described by John Hartwig and co-workers in the following references: (1) H. Morimoto, et al., “A Broadly Applicable Copper Reagent for Trifluoromethylations and Perfluoroalkylations of Aryl Iodides and Bromides,” Angew. Chem. Int. Ed., 2011, 50, 3793-98 W, (2) N. Litvinas, et al., “A General Strategy for the Perfluoroalkylation of Arenes and Aryl Bromides by Using Arylboronate Esters and [(phen)CuR^{F}],” Angew. Chem. Int. Ed., 2011, 50, 1-5, and (3) P. Fier, et al., “Copper Mediated Difluoromethylation of Aryl and Vinyl Iodides,” J. Am. Chem. Soc., published on the Web on Mar. 7, 2012. The disclosures of all three references are incorporated herein in their entirety. Thus, in one embodiment, an iodoarene is converted into a fluoroalkylarene using a copper reagent containing a fluoroalkyl group, X:

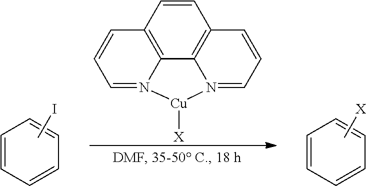

In an alternate embodiment, an arylboron substrate or, in favorable cases, a bromoarene, is used in place of an iodoarene. Although initially exemplified for the introduction of a trifluoromethyl group into arenes, the Hartwig technology is used in the practice of the present invention to introduce a difluoromethyl or C₂ to C₅ perfluoroalkyl group into an arene to prepare a compound of formula (1) as described herein.

Hartwig fluoroalkylation reagents having a C₂ to C₅ perfluoroalkyl group are prepared by reaction of the appropriate perfluoroalkyltrimethylsilane with copper (I) tert-butoxide in the presence of 1,10-phenanthroline. Some perfluoroalkyltrimethylsilanes are commercially available, for example, trifluoromethyltrimethylsilane “TMSCF₃” (also known as “Ruppert's reagent” and “Ruppert-Prakash reagent”) and perfluoroethyltrimethylsilane (“TMSCF₂CF₃”). Others are prepared in a manner analogous to the preparation of Ruppert's reagent, namely reaction of trimethylsilyl chloride with the appropriate perfluorobromide (CF₃CF₂Br, CF₃CF₂CF₂Br, CF₃CF₂CF₂CF₂Br, CF₃CF₂CF₂CF₂CF₂Br, in tris-(diethylamino)phosphine or other suitable solvent:

A Hartwig fluoroalkylation reagent containing the difluoromethyl group, —CHF₂, can be generated in situ by reacting trimethylsilyl difluoromethane (“TMSCF₂H”), copper (I) iodide, and cesium fluoride, and then used to difluoromethylate an iodo arene. TMSCF₂H can be prepared in 70% yield by reacting trifluorotrimethylsilane with sodium borohydride in diglyme at room temperature.

Compounds of formula (1) are prepared by introducing a difluoromethyl or C₂ to C₅ perfluoroalkyl group early in the synthesis of a target compound, or by introducing a difluoromethyl or C₂ to C₅ perfluoroalkyl group later in the synthesis by replacement of an appropriately positioned iodo, bromo, or boron group (i.e., a boronate ester) in the molecule. Schematically, these two general approaches can be depicted as follows:

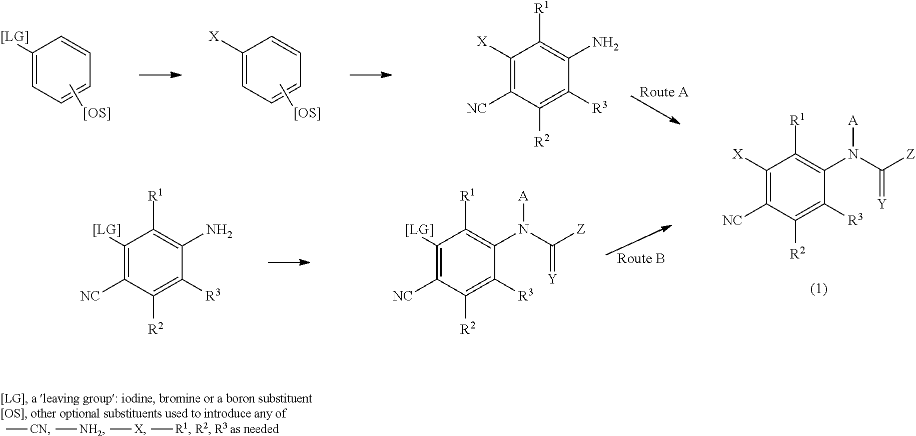

In Route A, compounds of formula (1) are prepared using any of various reaction sequences in which one step is the transformation of a suitable iodoarene intermediate into the corresponding compound in which the fluoroalkyl substituent has replaced the iodine atom. For example, in one embodiment, leaving group [LG] is iodine, X is pentafluoroethyl, and the first step is the conversion of iodobenzene into a pentafluoroethylbenzene. In an alternate embodiment, an arylboron substrate or, in favorable cases, a bromoarene substrate, is used in place of an iodoarene.

In a variation of the above two general schemes, the group labeled ‘A’ is already bound to the amino group when the leaving group [LG] is converted into the fluoroalkyl substituent X.

In carrying out the fluoroalkylation reaction on an iodoarene (or similar substrate) bearing an amino group, the amino group is typically protected with a suitable protecting group prior to reaction with the desired phenanthroline copperfluoroalkyl reagent. Protecting groups useful for protection of amines in the practice of the fluoroalkylation chemistry include t-butyloxycarbonyl (“BOC”), carboxybenzyl (also known as benzyloxycarbonyl, “CBZ,” and “Z”), fluorenylmethyloxycarbonyl (FMOC), acetyl, formyl, benzoyl, tosyl, and similar groups well known to those skilled in the art. These protecting groups may also be removed, if desired or necessary, under deprotection conditions appropriate for the protecting group used. For example, BOC may be removed with acids such as HCl; CBZ or Z may be removed by hydrogenolysis under mild conditions; FMOC may be removed by treatment with a mild base such as piperidine; acetyl, benzoyl, formyl, or tosyl may be removed by acid or base-catalyzed hydrolysis. In some cases, the precursor of the iodoarene (or similar substrate) bearing an amino group will be the corresponding iodoarene (or similar substrate) bearing a nitro group. In that instance, a preferred option is first to carry out the fluoroalkylation reaction on the substrate bearing the nitro group, and second to reduce that nitro group to an amino group.

Similarly, in carrying out the fluoroalkylation reaction on an iodoarene (or similar substrate) bearing a hydroxyl group, in particular a phenol, the hydroxyl group may need to be protected with a suitable protecting group prior to reaction with the desired phenanthroline copperfluoroalkyl reagent. Groups for protecting hydroxyl groups are well known in the art, and include, for example, acetyl, benzoyl, benzyl, trityl, dimethoxytrityl, methoxymethyl, trimethylsilyl, and the like. Deprotecting conditions for removing these groups are similarly well known in the art.

In the various reaction schemes that follow, when compounds bearing amino or hydroxyl groups are reacted with the fluoralkylation reagents, the steps of protection and deprotection, as appropriate, are assumed to be included even though they may not be mentioned specifically in every case. However, without being bound by theory, it is believed that, with some substrates, under certain reaction conditions, a protecting group will not be required.

The following six primary routes, with additional variations as provided herein, are representative of the synthetic methodologies that can be employed to prepare compounds of formula (1). The description initially focuses on those compounds in which each of R¹, R², R³, and A is hydrogen.

According to a first synthetic route, iodobenzene is converted into the corresponding difluoromethylbenzene or C₂ to C₅ perfluoroalkylbenzene, and then subsequent synthesis follows the steps known when starting with benzotrifluoride, but using the different fluoroalkylbenzene instead. This route is represented by the following scheme:

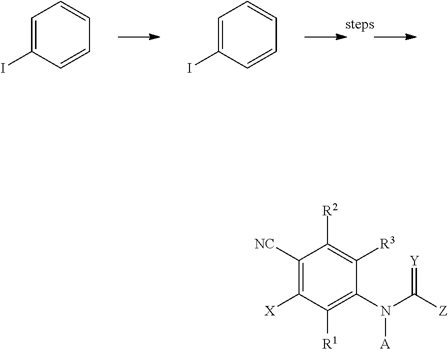

For example, a fluoroalkylbenzene is prepared from iodobenzene and then transformed by a series of reactions for converting it into a target compound, as described starting with benzotrifluoride in U.S. Pat. No. 3,012,058; that is, chlorination to the 1-(fluoroalkyl)-3-chlorobenzene; nitration and reduction to the 2-(fluoroalkyl)-4-chloroaniline; diazotization and treatment with copper cyanide to provide the 2-(fluoroalkyl)-4-chlorobenzonitrile; displacement of the chlorine atom with ammonia to provide the 2-(fluoroalkyl)-4-aminobenzonitrile; and acylation of the amino group to provide a compound of formula (1) that is an analogue of bicalutamide.

As an alternative, following the methodology for converting benzotrifluoride into a target compound, as described in EP0002892 (the disclosure of which is incorporated by reference herein in its entirety), the fluoroalkylbenzene is nitrated and the nitro group is reduced to yield the 3-(fluoroalkyl)aniline, which is brominated to give the 3-(fluoroalkyl)-4-bromoaniline, and the bromine atom is displaced by copper cyanide in dimethylformamide (DMF) to give the 2-(fluoroalkyl)-4-aminobenzonitrile, which is acylated to provide a compound of formula (1) that is an analogue of bicalutamide.

Compounds of formula (1) where Z is a substituted or unsubstituted alkyl group may be obtained through acylation of the arylamine intermediates by using conventional organic chemistry, for example by reaction with an acyl chloride (e.g., in a two-phase mixture containing aqueous base according to the Schotten-Baumann procedure). Compounds of formula (1) where Z is a substituted or unsubstituted amino group may be obtained for example by either (i) converting the arylamine intermediate into the isocyanate using phosgene, diphosgene, triphosgene, or the like, and then allowing that isocyanate to react with an amine representing the Z group, or (ii) allowing the arylamine intermediate to react with an isocyanate or alternative carbamoylating compound.

Compounds of formula (1) in which Y is sulfur may be made analogously to the compounds where Y is oxygen by substituting a relevant thiocarbonyl reagent; for example by treating the arylamine intermediate with a thioacyl chloride, RC(═S)Cl, e.g., (CH₃)₂CH₂C(═S)Cl, or an isothiocyanate RN═C═S, or alternatively converting the arylamine intermediate into the isothiocyanate using thiophosgene, and then allowing that isothiocyanate to react with an amine representing the Z group.

Compound of formula (1) in which Y is sulfur may also be made in favorable cases from the corresponding compounds in which Y is oxygen by a reaction that exchanges oxygen for sulfur, for instance by reaction with phosphorus pentasulfide or Lawessons's reagent, as described in Organic Syntheses, Collective Volume 7, 372 (1990).

Compounds of formula (1) in which A is a C₁-C₄ alkyl group may be made by way of either (i) conversion of the arylamine intermediate into the aryl(N-alkyl)intermediate, for example by forming an imine from condensation with an aldehyde and then reduction with sodium cyanoborohydride, or (ii) alkylation of the nitrogen atom from the arylamine intermediate after it has been acylated, thioacylated, aminocarbonylated, or aminothiocarbonylated, as the case may be, for example by treatment with base and an alkylating agent such as iodoethane. Compounds of formula (1) in which A is an acyl group may be made by an additional acylation of the nitrogen atom, either before or after the acylation, thioacylation, aminocarbonylation, or aminothiocarbonylation, as the case may be to introduce the Z group. Compounds of formula (1) in which A together with Z forms a cyclic group may be obtained by using a species that has the necessary difunctionality to do so; for example if the alkyl group of Z additionally bears an acylating group then it may acylate the nitrogen atom derived from the arylamine intermediate intramolecularly to form a ring. Such ring-formation may be take place, for example, where the group Z bears a nitrile function. Nonlimiting examples of cyclic groups include imidazolidinyl, hydantoin, and thiohydantoin.

A second route for providing compounds of formula (1) starts with 3-iodo-4-nitroaniline:

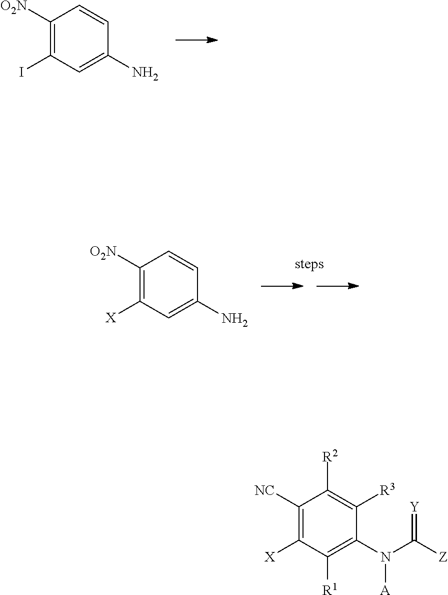

3-Iodo-4-nitroaniline is commercially available or obtained by nitration of 3-iodoaniline. Treatment with a Hartwig reagent yields 3-(fluoroalkyl)-4-nitroaniline, which can then be acylated, the nitro group reduced to an amino group then transformed into its diazonium salt with nitrous acid, and the resulting intermediate treated with copper cyanide to provide a compound of formula (1) that is an analogue of bicalutamide.

A third route for providing compounds of formula (1), applicable in the case where W is cyano, starts with 2-iodo-4-aminobenzonitrile, which is a known compound obtainable from commercially available 2-amino-4-nitrobenzoic acid by the sequence of reactions described in M. E. Van Dort et al, J. Med. Chem., 2000, 43, 3344-47 (incorporated by reference herein in its entirety). This is transformed into the corresponding 2-(fluoroalkyl)-4-aminobenzonitrile (with protectiondeprotection of the amino group as described above when required), and the amino group is derivatized to give a compound of formula (1) according to the scheme below.

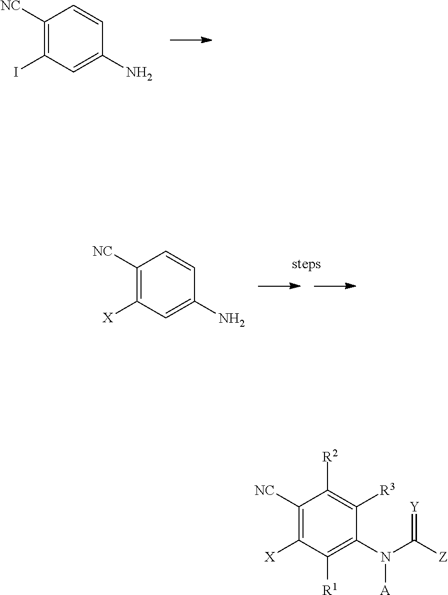

A fourth route for providing compounds of formula (1) starts with the commercially available 2-iodo-4-nitroaniline. The iodine atom is replaced by the fluoroalkyl group, using a Hartwig reagent, with protectiondeprotection of the amino group as described above when required. The amino group is then transformed to the nitrile group via the diazonium salt, and the nitro group is then reduced to an amino group. In principle, these three steps can be conducted in any order. Finally the amino group is derivatized as required to provide a compound of formula (1):

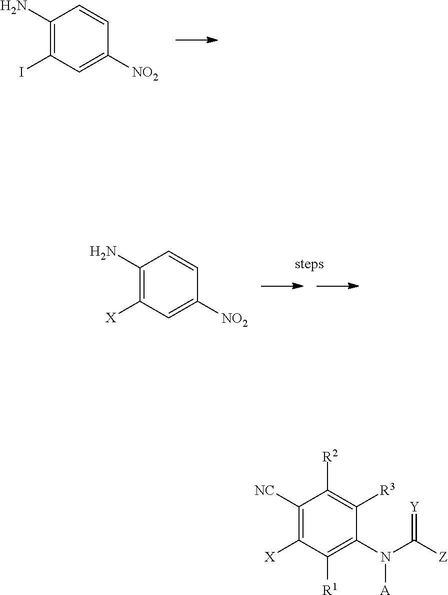

A fifth route for providing compounds of formula (1) starts with the commercially available 2-iodo-4-chloroaniline. According to the scheme below, the iodine atom is replaced by the fluoroalkyl group. Thereafter steps as described in U.S. Pat. No. 3,012,058 (incorporated by reference herein in its entirety) for the corresponding 2-(trifluoromethyl)-4-chloroaniline are followed, coinciding with steps of the first route above:

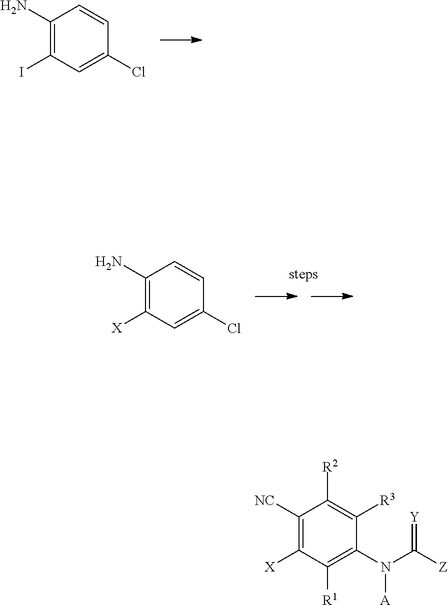

A sixth route for providing compounds of formula (1) starts with a suitable iodo compound that is or may be a selective androgen receptor modulator, and then the iodine is replaced by the fluoroalkyl group using the technology described herein, as depicted in the scheme below. Syntheses of iodo compounds that are selective androgen receptor modulators are described in M. E. Van Dort et al, J. Med. Chem., 2000, 43, 3344-47 and V. A. Nair et al, Tetrahedron Lett., 2004, 45, 9475-77 (the disclosures of which are incorporated by reference herein in their entirety).

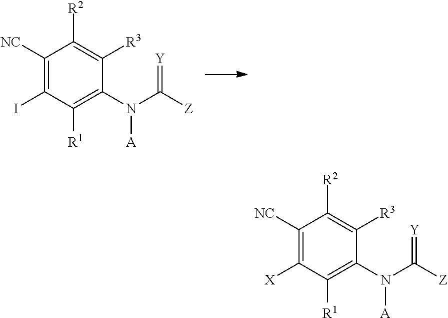

For each of the routes 1 to 6 described above, in the case where any of the groups R¹, R², and R³ are anything other than hydrogen, i.e., halogen or C₁ to C₆ alkyl, such substituents are either already present in an initial reactant (i.e., an R¹, R², and/or R³-substituted: iodobenzene (route 1), 3-iodo-4-nitroaniline (route 2), 2-iodo-4-aminobenzonitrile (route 3), 2-iodo-4-nitroaniline (route 4), 2-iodo-4-chloroaniline (route 5), or a compound having the formula:

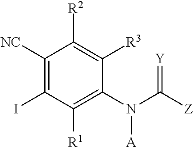

(route 6)) or introduced at some later point using methodologies for introducing halogen and/or alkyl groups known in the art of organic synthesis. For example, in one embodiment, appropriate aniline intermediates as described in the above routes are additionally halogenated ortho to the amino group. Iodination ortho to the amino group in these aniline compounds may be accomplished by methods known in the literature of organic synthesis; for example, using iodine monochloride as described in P. Block, J. Org. Chem., 1956, 21, 1237-39, or using iodine and silver sulfate as described by Wing-Wah Sy, Synth. Commun., 1992, 22, 3215. The latter paper gives, for example, the conversion of p-nitroaniline into 2-iodo-4-nitroaniline. A more recent method for the controlled iodination of anilines is in H. W. Mbatia et al, Org. Biomol. Chem., 2011, 9, 2987-91 using an 18-Crown-6 complex of potassium iododichloride.

In the case where the halogen is iodine, it can be replaced with a methyl, ethyl, or larger alkyl group using an alkyl-copper reagent, e.g., lithium dimethyl cuprate, such as indicated by the following scheme.

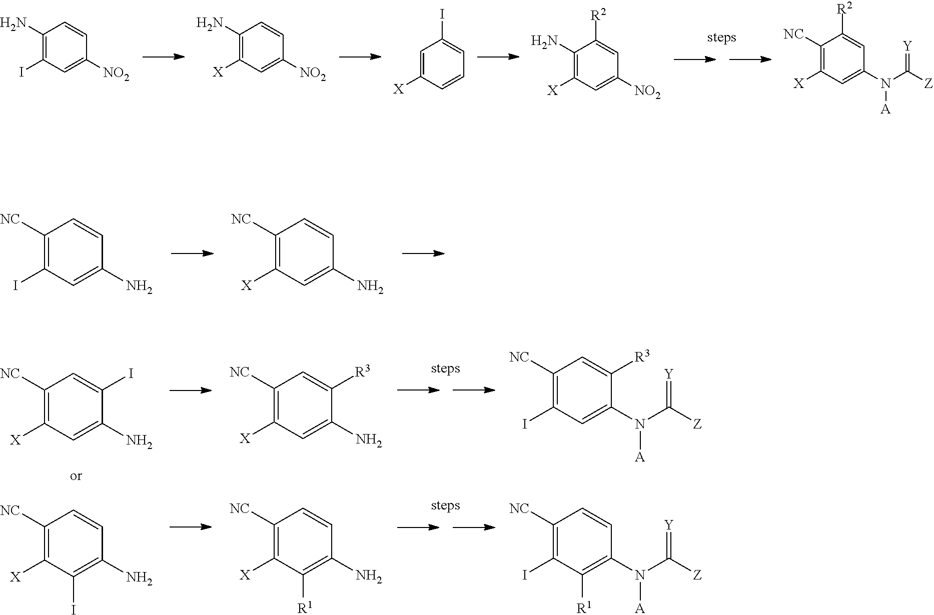

Compounds containing additional alkyl substitution, such as a methyl or ethyl group on the fluoroalkylaminobenzonitrile moiety may be accessed by a combination of known methods and as described herein. For example, in one approach, one starts with the appropriate iodoalkylbenzene, for example o-, m- or p-iodotoluene, and the iodine atom is then replaced by a difluoromethyl or C₂-C₅ fluoroalkyl group according to the methodology described herein. The resulting compounds can be nitrated and the nitro functions of the appropriate isomer reduced to afford, respectively, for example, the 2-methyl-3-fluoroalkylaniline, the 3-methyl-5-fluoroalkylaniline, and the 2-methyl-5-fluoroalkylaniline. Bromination and displacement of the bromine atom using copper (I) cyanide will afford a perfluoroalkylaminobenzonitrile suitable as an intermediate to obtain compounds of this invention. One such reaction sequence is given by the following scheme:

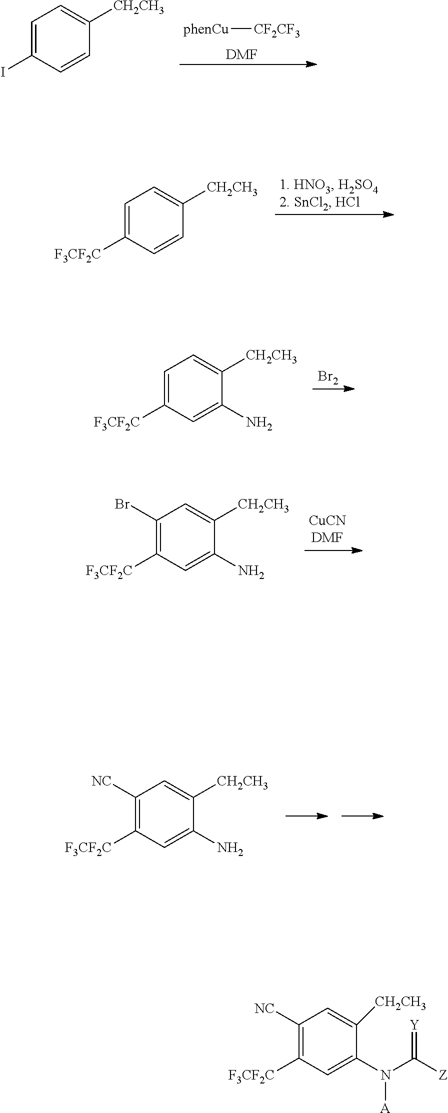

As an alternative approach, alkyl groups may be introduced onto the benzene ring by way of displacement of iodine in an iodoarene using an alkylcopper reagent such as lithium dimethylcuprate, as described in G. M. Whitesides et al, J. Am. Chem. Soc., 1969, 91, 4871-82. Lithium dimethylcuprate can be used to introduce an ethyl group into the compound. Accordingly a methyl, ethyl, or higher alkyl group may be introduced either before or after the fluoroalkyl group, for example starting with 2-iodo-4-nitroaniline in accordance with the scheme below (showing the introduction of a methyl group):

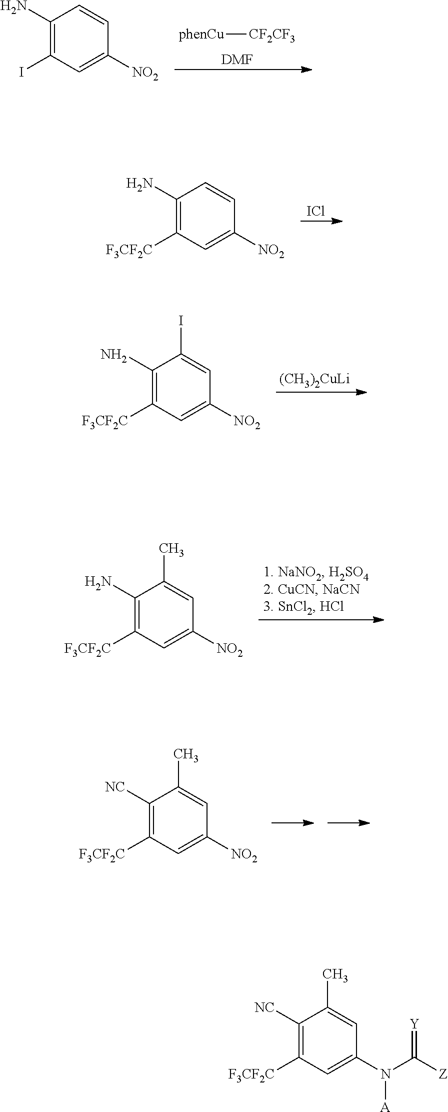

Compounds containing additional halogen substitution, such as a fluorine, chlorine, or bromine atom attached to the fluoroalkylaminobenzonitrile moiety may be obtained by analogous routes to the above, either by starting with an appropriate halogenobenzene derivative, such as 1-fluoro-4-iodobenzene, instead of the corresponding alkylbenzene compound, or by introducing a different halogen directly by treatment with a halogenating agent such as N-chlorosuccinimide or bromine.

With regard to the nitrogen-bearing side chain having the substituents A, Y, and Z in the compounds of formula (1), in one embodiment of the invention the side chain is constructed using synthetic methodologies previously used to prepare compounds that possess a trifluoromethyl group, but using a difluoromethyl or C₂ to C₅ perfluoroalkyl group instead. Such methodologies are described, for example, in U.S. Pat. No. 3,995,060, U.S. Pat. No. 4,636,505, US 2007254,933, and U.S. Pat. No. 6,569,896, the disclosures of which are incorporated by reference herein in their entirety. Specific examples are provided below.

The following schemes are representative of the methodologies for acylating the amino functionality, introducing a group other than hydrogen onto the amide nitrogen (group “A” in formula (1)), and forming a cyclic group with A and Z. Here, X is difluoromethyl or C₂ to C₅ perfluoroalkyl, or X is another substituent, such as iodine, that can be transformed into said difluoromethyl or C₂ to C₅ perfluoroalkyl group after the following transformations, or within the sequence if the transformation has more than one step.

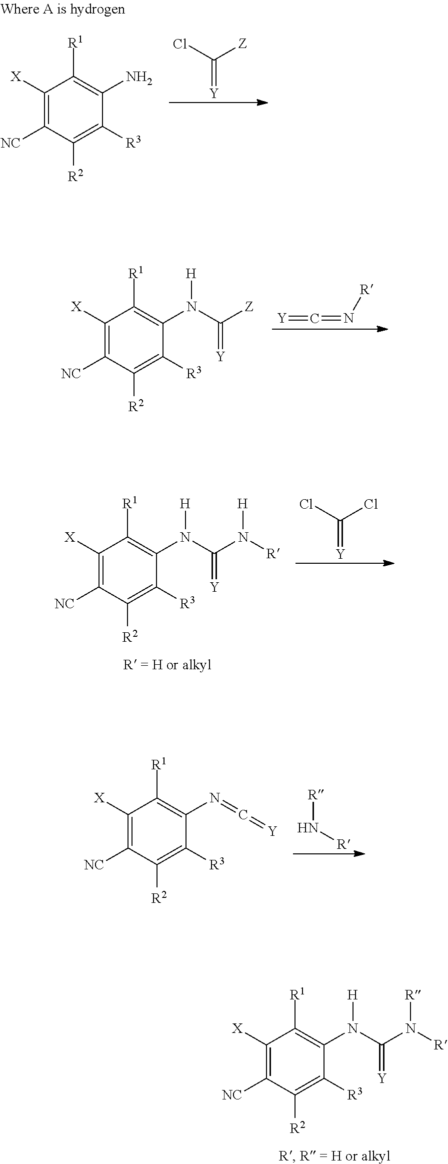

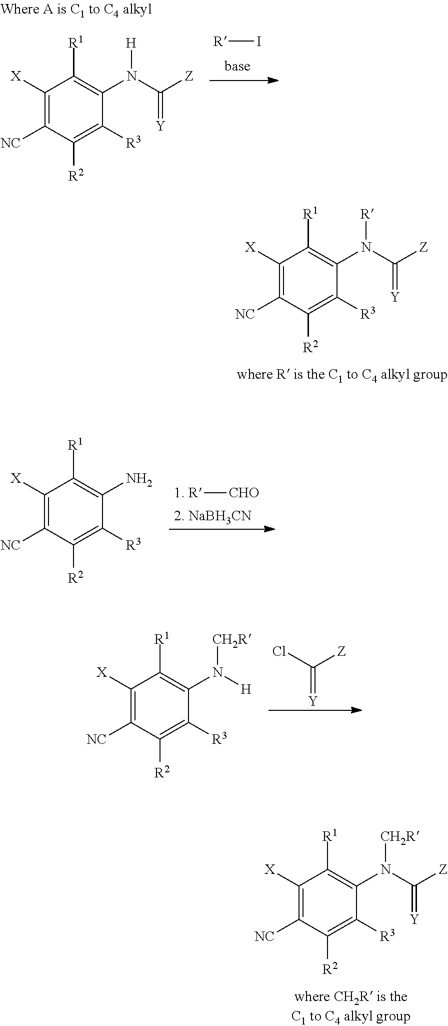

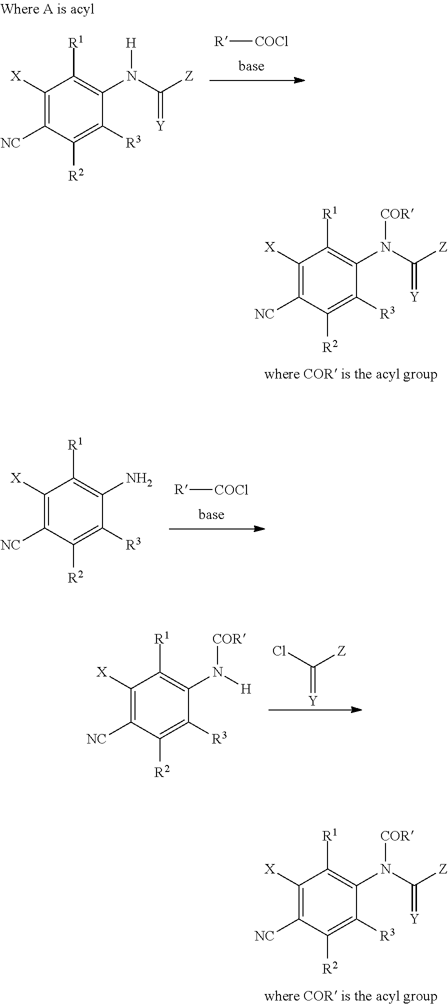

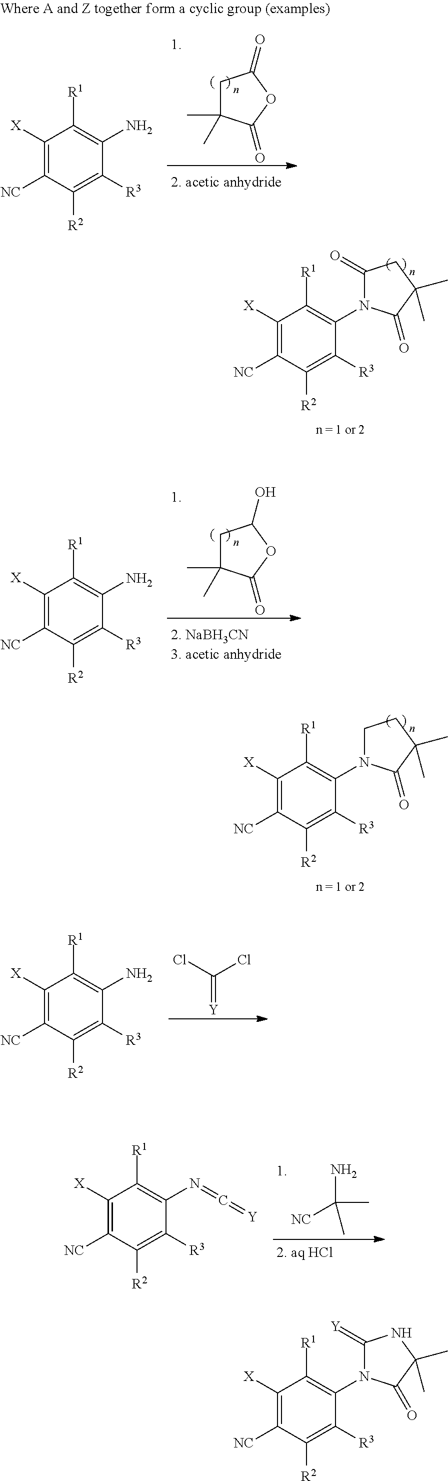

The following are nonlimiting examples of the invention.

### Example 1

### N-(4-Cyano-3-(perfluoropentyl)phenyl)isobutyramide

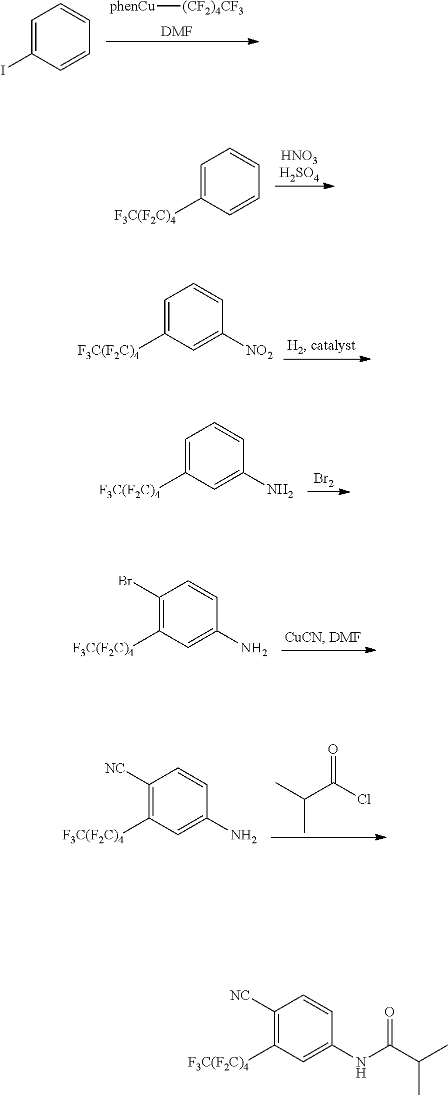

The fluoroalkylation reagent is prepared by the reaction of copper (I) t-butoxide with 1,10-phenanthroline followed by the addition of perfluoropentyltrimethylsilane, analogous to the method described in J. F. Hartwig et al, Angew. Chem. Int. Ed., 2011, 50, 1-7 for the preparation of the corresponding perfluoropropyl reagent. A mixture of the obtained perfluoropentylcopper phenanthroline complex (1.5 equivs) and iodobenzene in N,N-dimethylformamide (DMF) is heated at 50° C. for 18 hours to afford (perfluoropentyl)benzene. Nitration may be conducted with a mixture of nitric and sulfuric acids or for example as described in U.S. Pat. No. 3,714,272 for benzotrifluoride wherein a solution of the (perfluoropentyl)benzene with 4 equiv. anhydrous nitric acid and 8 equiv. trifluoromethansulfonic acid in dichloromethane after 16 hrs at 25° C. affords nitro products, of which over 90% is the required meta isomer. The obtained 3-nitro-(perfluoropentyl)benzene is reduced under 10 atmospheres of hydrogen in methanol solution using 10 wt % Raney nickel catalyst over 40 hours, such as by the method described in U.S. Pat. No. 6,333,434, to provide the 3-(perfluoropentyl)aniline. Bromination is carried out using bromine, or N-bromosuccinimide as described in Bartoli et al, Synthesis, 2009, 1305-08 to give 3-(perfluoropentyl)-4-bromoaniline. Displacement of the bromine by cyanide is undertaken by heating with copper (I) cyanide in boiling N,N-dimethylformamide as described in EP0002892. Finally, the obtained 2-(perfluoropentyl)-4-aminobenzonitrile is acylated with isobutyryl chloride in pyridine, such as by the method described in U.S. Pat. No. 3,995,060 on related compounds, to afford the N-(4-Cyano-3-(perfluoropentyl)phenyl)isobutyramide.

### Example 2

### Example 2. N-(4-Cyano-3-(perfluoroethyl)phenyl)-3-(4-fluorophenylsulfonyl)-2-hydroxy-2-methylpropanamide

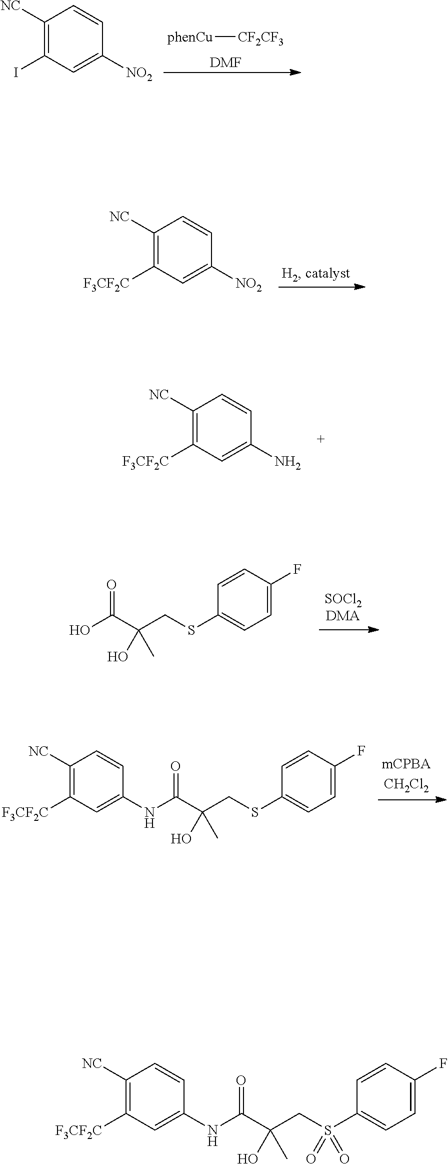

The fluoroalkylation reagent is prepared by the reaction of copper (I) butoxide with 1,10-phenanthroline followed by the addition of pentafluoroethyltrimethylsilane, analogous to the method described in J. F. Hartwig et al, Angew. Chem. Int. Ed., 2011, 50, 1-7 for the preparation of the corresponding perfluoropropyl reagent. A mixture of the obtained pentafluoroethylcopper phenanthroline complex (1.5 equivs) and 4-nitro-2-iodobenzonitrile, obtained as described in V. A. Nair et al., Tetrahedron Lett., 2004, 45, 9475-77, in N,N-dimethylformamide (DMF) is heated at 50° C. for 18 hours. The obtained 4-nitro-2-(pentafluoroethyl)benzonitrile is reduced under 10 atmospheres of hydrogen in methanol solution using 10 wt % Raney nickel catalyst over 40 hours, such as by the method described in U.S. Pat. No. 6,333,434, to provide the 4-amino-2-(pentafluoroethyl)benzonitrile. To a solution of 3-(4-fluorophenylthio)-2-hydroxy-2-methylpropanoic acid, obtained as described in U.S. Pat. No. 4,636,505, in N,N-dimethylacetamide (DMA) at −15° C. is added thionyl chloride over 15 min, then the 4-amino-2-(pentafluoroethyl)benzonitrile is added. After 30 min at −15° C. the mixture is allowed to warm to room temperature for 15 hours, after which the N-(4-cyano-3-(perfluoroethyl)phenyl)-3-(4-fluorophenylthio)-2-hydroxy-2-methylpropanamide is recovered. To a solution of this sulfide in dichloromethane is added a solution of m-chloroperoxybenzoic acid (mCPBA) in dichloromethane over 30 mins and the mixture is stirred at room temperature for 18 hours, after which the title sulfone N-(4-cyano-3-(perfluoroethyl)phenyl)-3-(4-fluorophenylsulfonyl)-2-hydroxy-2-methylpropanamide is recovered.

### Example 3

### 4-(3-(4-Cyano-3-(perfluoropropan-2-yl)phenyl)-5,5-dimethyl-4-oxo-2-thioxoimidazolidin-1-yl)-2-fluoro-N-methylbenzamide

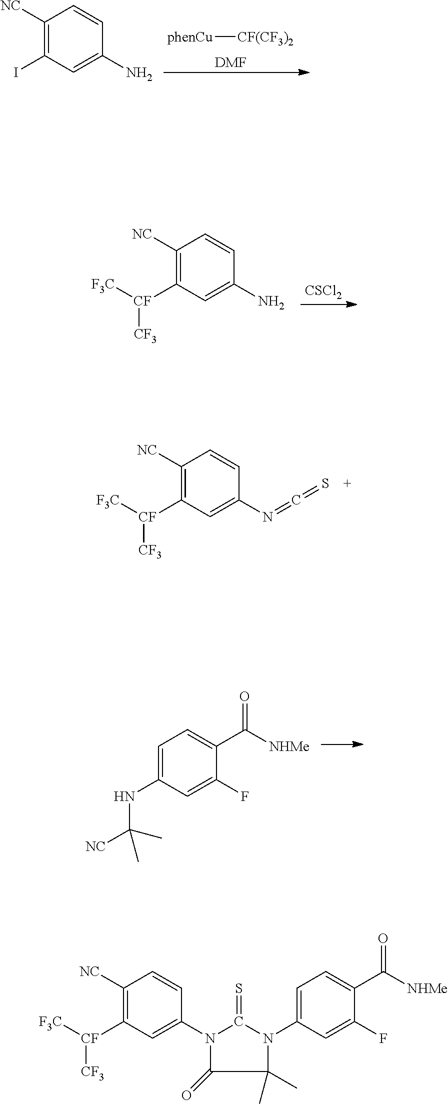

The fluoroalkylation reagent is prepared by the reaction of copper (I) butoxide with 1,10-phenanthroline followed by the addition of perfluoroisopropyltrimethylsilane, analogous to the method described in J. F. Hartwig et al, Angew. Chem. Int. Ed., 2011, 50, 1-7 for the preparation of the corresponding perfluoro-n-propyl reagent. A mixture of the obtained perfluoroisopropylcopper phenanthroline complex (1.5 equivs) and 4-amino-2-iodobenzonitrile, obtained as described in V. A. Nair et al., Tetrahedron Lett., 2004, 45, 9475-77, in N,N-dimethylformamide is heated at 50° C. for 18 hours to afford 4-amino-2-(perfluoroisopropyl)benzonitrile. The following steps follow the method described in US2007254933. Thus the aminobenzonitrile is added over 15 min into a stirred two-phase mixture of thiophosgene (1.08 equiv) and water, and the resulting mixture is stirred a further 1 hour, from which the crude 4-isothiocyanato-2-(perfluoroisopropyl)benzonitrile is isolated. It is dissolved in N,N-dimethylformamide, 4-(2-cyanopropan-2-ylamino)-2-fluoro-N-methylbenzamide is added, and the mixture is heated at 100° C. for 11 hours. Methanol and 1 M aqueous hydrochloric acid are added and the mixture heated at reflux for 1.5 hours, from which the title compound 4-(3-(4-cyano-3-(perfluoropropan-2-yl)phenyl)-5,5-dimethyl-4-oxo-2-thioxoimidazolidin-1-yl)-2-fluoro-N-methylbenzamide is subsequently isolated.

### Example 4

### (S)—N-(4-Cyano-3-(difluoromethyl)phenyl)-3-(4-fluorophenoxy)-2-hydroxy-2-methylpropanamide

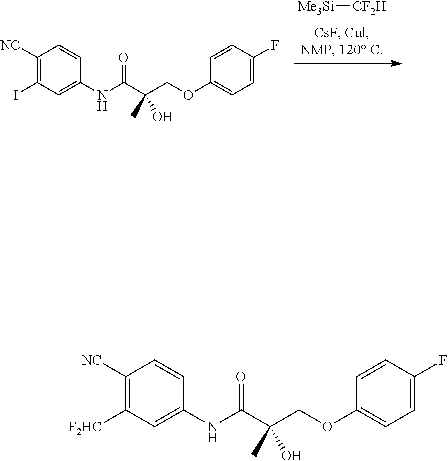 [SMILES: C[Si](C)(C)C(F)F ; C[C@](O)(COc1ccc(F)cc1)C(=O)Nc1ccc(C#N)c(I)c1 ; C[C@](O)(COc1ccc(F)cc1)C(=O)Nc1ccc(C#N)c(C(F)F)c1]

(S)—N-(4-Cyano-3-iodophenyl)-3-(4-fluorophenoxy)-2-hydroxy-2-methylpropanamide is prepared as described in V. A. Nair et al., Tetrahedron Lett., 2004, 45, 9475-77. Its conversion into the title compound follows the fifluoromethylation method described in P. S. Fier and J. F. Hartwig, J. Am. Chem. Soc., 2012, in the press. Thus the iodophenyl compound (440 mg) is heated with (trifluoromethylsilyldifluoromethane (5 equiv, 621 mg, obtained by sodium borohydride reduction of trifluoromethylsilyltrifluoromethane in diglyme at ambient temperature), cesium fluoride (3 equiv, 456 mg), and copper (I) iodide (1 equiv, 190 mg) in N-methylpyrrolidinone at 120° C. for 24 h, to afford the title difluoromethyl compound.

From the preceding disclosure, various modifications and alternate embodiments of the invention will be apparent to persons skilled in the art to which the invention pertains. All such modifications and embodiments are within the scope of the invention, which is limited only by the appended claims and equivalents thereof.

1. A compound represented by the formula (1) 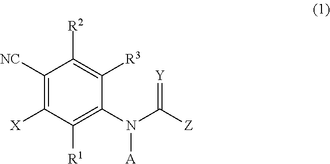
wherein
X is difluoromethyl or C₂ to C₅ perfluoroalkyl;
Y is oxygen or sulfur;
Z is selected from the group consisting of
(i) C₁ to C₆ alkyl, which may be linear or branched,
(ii) C₃ to C₆ cycloalkyl,
(iii) a five- or six-membered aryl group,
(iv) C₁ to C₆ alkoxy,
(v) C₃ to C₆ cycloalkoxy,
(vi) aryloxy wherein the aryl group is five- or six-membered,
(vii) amino,
(viii) alkylamino wherein the alkyl group is of 1 to 6 carbon atoms and may be cycloalkyl,
(ix) dialkylamino wherein both alkyl groups are of 1 to 6 carbon atoms, and either or both may be cycloalkyl,
(x) arylamino wherein the aryl group is five- or six-membered,
(xi) diarylamino wherein both aryl groups are five- or six-membered,
(xii) arylalkylamino wherein the aryl group is five- or six-membered and the alkyl group is of 1 to 6 carbon atoms and may be cycloalkyl, and
wherein in each case (i) to (xii) said alkyl, cycloalkyl, alkoxy or aryl group optionally bears one or more groups selected from hydroxy, alkoxy (—OR), aryloxy (—OAr), arylthio (—SAr), arylsulfoxide (—SOAr), and arylsulfone (—SO₂Ar), wherein each R group is independently selected from C1 to C4 alkyl, each Ar group is independently a five- or six-membered ring, and each R and each Ar group optionally bears one or more substituents selected from halogen, carboxamide, where the nitrogen atom optionally bears one or more C₁-C₄ alkyl groups, acylamino, where the acyl group contains 1 to 4 carbon atoms, and nitrile;
R¹, R², and R³ are, independently, selected from the group consisting of hydrogen, halogen, and C₁ to C₆ alkyl; and
A is hydrogen, C₁ to C₄ alkyl, or acyl, or A together with Z forms a five- or six-member heterocyclic group that optionally bears one or more groups selected from hydroxy, alkoxy (—OR), aryloxy (—OAr), arylthio (—SAr), arylsulfoxide (—SOAr), and arylsulfone (—SO₂Ar), wherein each R group is independently selected from C1 to C4 alkyl, each Ar group is independently a five- or six-membered ring, and each R and each Ar group optionally bears one or more substituents selected from halogen, carboxamide (where the nitrogen atom optionally bears one or more C₁-C₄ alkyl groups), acylamino (where the acyl group contains 1 to 4 carbon atoms), and nitrile.

2. A compound according to claim 1, wherein R¹, R², and R³ are hydrogen.

3. A compound according to claim 1, wherein X is pentafluoroethyl, hepatafluoropropyl, or heptafluoroisopropyl.

4. A compound according to claim 1, wherein X is pentafluoroethyl, and the compound has formula (2):

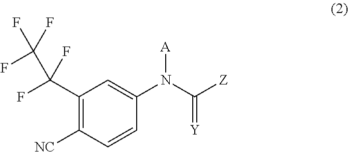

5. A compound according to claim 1, wherein A together with Z forms a five- or six-member heterocyclic group selected from the group consisting of imidazolidine-2,4-dione, 2-thioxoimidazolidin-4-one, 5-thioxoimidazolidin-2-one, imidazolidin-2-one, imidazolidine-2-thione, pyrrolidin-2-one, pyrrolidine-2-thione, oxazolidin-2-one, oxazolidine-2-thione, oxazolidine-2,4-dione, 1,2,4-oxadiazolidine-3,5-dione, 1,2,4-triazolidine-3,5-dione, 3-thioxo-1,2,4-oxadiazolidin-5-one, 5-thioxo-1,2,4-triazolidin-3-one, tetrahydropyrimidin-2(1H)-one, tetrahydropyrimidin-2(1H)-thione, piperidin-2-one, 1,3-oxazinan-2-one, 1,3-oxazinane-2,4-dione, piperidine-2,6-dione, dihydropyrimidine-2,4(1H,3H)-dione, and 2-thioxotetrahydropyrimidine-4(1H)-one.

6. A compound according to claim 5, wherein the five- or six-member heterocyclic group formed by A together with Z bears one or more groups selected from hydroxy, alkoxy (—OR), aryloxy (—OAr), arylthio (—SAr), arylsulfoxide (—SOAr), and arylsulfone (—SO₂Ar), wherein each R group is independently selected from C1 to C4 alkyl, each Ar group is independently a five- or six-membered ring, and each R and each Ar group optionally bears one or more substituents selected from halogen, carboxamide (where the nitrogen atom optionally bears one or more C₁-C₄ alkyl groups), acylamino (where the acyl group contains 1 to 4 carbon atoms), and nitrile.

7. A compound according to claim 1, having any one of the following formulas:

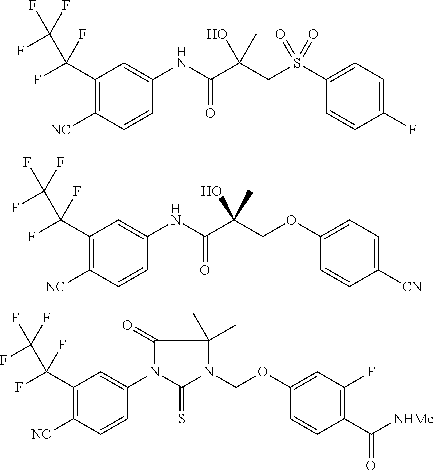

8. A compound according to claim 1, having the formula:

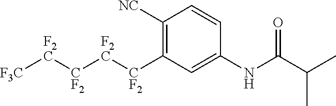 [SMILES: CC(C)C(=O)Nc1ccc(C#N)c(C(F)(F)C(F)(F)C(F)(F)C(F)(F)C(F)(F)F)c1]

9. A compound according to claim 1, having the formula:

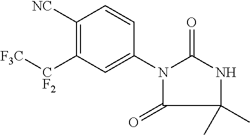 [SMILES: CC1(C)NC(=O)N(c2ccc(C#N)c(C(F)(F)C(F)(F)F)c2)C1=O]

10. A compound according to claim 1, having the formula:

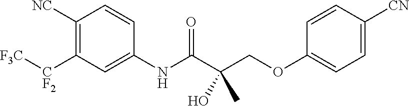 [SMILES: C[C@](O)(COc1ccc(C#N)cc1)C(=O)Nc1ccc(C#N)c(C(F)(F)C(F)(F)F)c1]

11. A compound according to claim 1, having the formula:

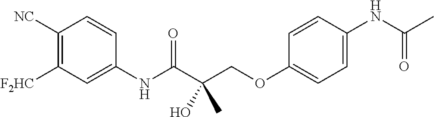 [SMILES: CC(=O)Nc1ccc(OC[C@](C)(O)C(=O)Nc2ccc(C#N)c(C(F)F)c2)cc1]

12. A compound according to claim 1, having the formula:

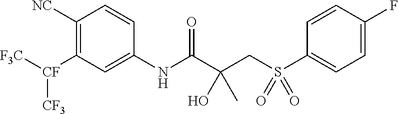

13. A compound according to claim 1, having the formula:

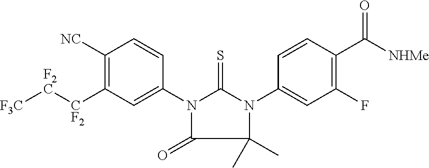 [SMILES: CNC(=O)c1ccc(N2C(=S)N(c3ccc(C#N)c(C(F)(F)C(F)(F)C(F)(F)F)c3)C(=O)C2(C)C)cc1F]

14. A compound comprising a physiologically acceptable salt of a compound recited in claim 1.

15. A compound of formula (1) as recited in claim 1, prepared by the process of:
forming a difluoromethylarene or C₂ to C₅ perfluoroalkylarene by treating an iodoarene with a Hartwig fluoroalkylation reagent having a difluoromethyl group or C₂ to C₅ perfluoroalkyl group; and
converting the difluoromethylarene or C₂ to C₅ perfluoroalkylarene into the compound of formula (1);
wherein the iodoarene is selected from the group consisting of

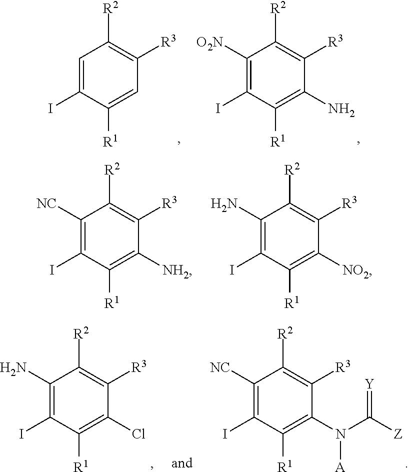

16. A process for preparing a compound represented by the formula (1) 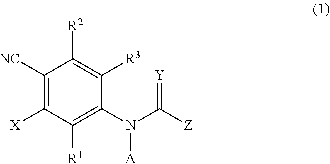
wherein
X is difluoromethyl or C₂ to C₅ perfluoroalkyl;
Y is oxygen or sulfur;
Z is selected from the group consisting of
(i) C₁ to C₆ alkyl, which may be linear or branched,
(ii) C₃ to C₆ cycloalkyl,
(iii) a five- or six-membered aryl group,
(iv) C₁ to C₆ alkoxy,
(v) C₃ to C₆ cycloalkoxy,
(vi) aryloxy wherein the aryl group is five- or six-membered,
(vii) amino,
(viii) alkylamino wherein the alkyl group is of 1 to 6 carbon atoms and may be cycloalkyl,
(ix) dialkylamino wherein both alkyl groups are of 1 to 6 carbon atoms, and either or both may be cycloalkyl,
(x) arylamino wherein the aryl group is five- or six-membered,
(xi) diarylamino wherein both aryl groups are five- or six-membered,
(xii) arylalkylamino wherein the aryl group is five- or six-membered and the alkyl group is of 1 to 6 carbon atoms and may be cycloalkyl, and
wherein in each case (i) to (xii) said alkyl, cycloalkyl, alkoxy or aryl group optionally bears one or more groups selected from hydroxy, alkoxy (—OR), aryloxy (—OAr), arylthio (—SAr), arylsulfoxide (—SOAr), and arylsulfone (—SO₂Ar), wherein each R group is independently selected from C1 to C4 alkyl, each Ar group is independently a five- or six-membered ring, and each R and each Ar group optionally bears one or more substituents selected from halogen, carboxamide, where the nitrogen atom optionally bears one or more C₁-C₄ alkyl groups, acylamino, where the acyl group contains 1 to 4 carbon atoms, and nitrile;
R¹, R², and R³ are, independently, selected from the group consisting of hydrogen, halogen, and C₁ to C₆ alkyl; and
A is hydrogen, C₁ to C₄ alkyl, or acyl, or A together with Z forms a five- or six-member heterocyclic group that optionally bears one or more groups selected from hydroxy, alkoxy (—OR), aryloxy (—OAr), arylthio (—SAr), arylsulfoxide (—SOAr), and arylsulfone (—SO₂Ar), wherein each R group is independently selected from C1 to C4 alkyl, each Ar group is independently a five- or six-membered ring, and each R and each Ar group optionally bears one or more substituents selected from halogen, carboxamide (where the nitrogen atom optionally bears one or more C₁-C₄ alkyl groups), acylamino (where the acyl group contains 1 to 4 carbon atoms), and nitrile, comprising:
firming a difluoromethylarene or C₂ to C₅ perfluoroalkylarene by treating an iodoarene with a Hartwig fluoroalkylation reagent having a difluoromethyl group or C₂ to C₅ perfluoroalkyl group; and
converting the difluoromethylarene or C₂ to C₅ perfluoroalkylarene into the compound of formula (1):
wherein the iodoarene is selected from the group consisting of [chemical structure]
